(19) United States
(12) Patent Application Publication
(10) Pub. No.: US 2026/0174660 A1
(43) Pub. Date: Jun. 25, 2026

(54) UV FILTER COMPOSITION COMPRISING TRITERPENES, EMOLLIENTS AND INORGANIC UV-FILTERS

(71) Applicant: AAK AB (PUBL), Malmö (SE)

(72) Inventors: Benjamin C. SCHWARTZ, Malmö (SE); Staffan NORBERG, Malmö (SE)

(21) Appl. No.: 19/123,164

(22) PCT Filed: Oct. 25, 2023
(86) PCT No.: PCT/SE2023/051053

§ 371 Date: Apr. 22, 2025

(30) Foreign Application Priority Data
Oct. 25, 2022 (SE) 2230343-2

(51) Int. Cl. A61K 8/63; A61Q 17/04

(52) U.S. Cl. CPC A61K 8/63; A61Q 17/04; A61K 2800/31

(57) ABSTRACT

The present invention relates to a UV filter composition, and a formulation comprising said UV filter composition. The invention also relates the use of said UV filter composition in a formulation. In particular, the invention relates to the use of certain UV filter composition in a formulation to improve various properties such as the viscosity and flowability.

### TECHNICAL FIELD OF THE INVENTION

The present invention relates to a UV filter composition, and a formulation comprising said UV filter composition. The invention also relates to the use of said UV filter composition in a formulation. In particular, the invention relates to the use of certain UV filter composition in a formulation to improve various properties such as the viscosity and flowability.

### BACKGROUND OF THE INVENTION

A typical cosmetic and personal care formulation contains from 10 to 50 ingredients. Each ingredient contributes with its individual value to the formulation. The formulation is created to give specific values to the consumer.

Over the last years, cosmetic and personal care formulation such as sunscreen products have evolved considerably. Several aspects of these products have been improved such as the texture and the visual aspect. Sunscreen products generally absorb ultraviolet light (UV light), in particular UV (A) and UV (B). UV (A) filters absorb electromagnetic radiation of the wavelength between 320 and 400 nm. UV (B) filters absorb electromagnetic radiation of the wavelength between 290 and 320 nm.

Sunscreen products usually contain inorganic or organic UV filters. The process for producing sunscreen formulations including organic UV filters includes the dissolution of organic UV filters in a solvent before mixing them with the other ingredients. The process for producing inorganic UV filters-based formulation includes the dispersion of inorganic filters, such as titanium dioxide or zinc oxide, in the formulation. Nevertheless, since inorganic UV filters are in solid form, only a limited amount of inorganic UV filters may be incorporated in a sunscreen formulation whilst maintaining a homogenous and well dispersed formulation. Furthermore, sunscreen products such as sprayable sunscreens are difficult to produce when using increased quantities of inorganic UV filters.

It is known to use shea butter as vegetable oil in the field of cosmetics and personal care today. Shea Butter (Butyrospermum Parkii Butter) originates from the plant Vitellaria paradoxa. Shea Butter is semi-solid and non-fluid at room temperature. Shea Butter has a melting point between 31-42° C. as measured by AOCS Official Method Cc 3-25. Shea Butter mainly comprises triglycerides, in particular above 80 wt. %. Triglycerides are dominated by two main fatty acids: stearic fatty acid residues that can be present from 38 to 48 wt. % of the fatty acid composition and the oleic fatty acid residues that can be present from 44 to 54% of the fatty acid composition. The diglycerides amount may be typically between 2 and 6%. Shea butter also typically contains from 2 to 4 wt. % of triterpene esters and 2-4% of high molecular isoprenoid hydrocarbons called karitenes.

The liquid fraction of shea butter, also called shea butter oil or shea olein, stands out in the market of vegetable oils within the cosmetic market for several reasons. As a basic delivery medium it combines a good appearance with good oxidation stability. Shea olein (Butyrospermum parkii oil) may be defined as a liquid oil phase originating from the plant Vitellaria paradoxa. Shea olein is fluid at room temperature and has a melting point below 30° C. as measured by AOCS Official Method Cc 3-25. Shea olein comprises above 70 wt. % triglycerides dominated by two main fatty acids: the stearic fatty acid residues that can be present from 20 to 34% of the fatty acid composition and Oleic fatty acid residues that can be present from 44 to 60% of the fatty acid composition. The diglyceride level amount may be between 4 and 13%. Shea olein also contains from 6 to 13% of triterpene esters and typically less than 1% of high molecular isoprenoidal hydrocarbons called karitenes.

The main triterpene alcohols in shea butter and shea olein are lupeol, alpha-amyrin, beta-amyrin, butyrospermol and parkeol (Peers, J. Sci. Food Agric. 28 (11), 1000-1009, (1977). The triterpene alcohols are esterified with mainly three types of acids: long chain fatty acids, acetic acid and cinnamic acid. Around half of the triterpenes are esterified with cinnamic acid, e.g. around 4 wt. % of the shea olein contains triterpene cinnamates.

Such triterpene esters have been shown to provide anti-ageing and anti-inflammatory properties, often sought for within cosmetic applications (Akihisa et al J Oleo Sci, 59 (6), 273-280 (2010).

WO9963031 discloses the use of unsaponifiable lipids of shea butter comprising UV-absorbing triterpene esters and natural phytosterols to provide anti-inflammatory effects for damaged skin. The shea butter fraction can be used as an ingredient of cosmetic and pharmaceutical preparations, especially for providing UV-protecting and skin moisturizing properties. Example 11 discloses a UV-stick comprising 2% by weight of titanium oxide and octyl palmitate (Tioveil™ OP), 4% by weight of shea butter (Lipex® Shea-U™), 4% by weight of hydrogenated canola oil (AKOREX L), 23% by weight of caprylic/capric triglycerides (AKOMED®), 7% by weight of a hydrogenated vegetable oil (AKOICE E), 4% by weight of beeswax (Beeswax white), 6% by weight of candelilla wax, 15% by weight of palm kernel oil (LIPEX 403) and 35% by weight of a hydrogenated vegetable oil (LIPEX 408).

The present inventors have found that when using such composition, there is a risk that the viscosity is very high leading to high risks to produce a heterogenous and non-dispersed formulation. Indeed, despite the fact that the composition comprises a very low amount of UV filters and about 4% of Lipex® Shea-U™, i.e. about 1 wt. % of triterpenes alcohols and/or triterpenes esters, the flowability is very low and the viscosity is too high to obtain a well dispersed formulation.

There remains a need for UV filter compositions that seek to solve or alleviate many of the problems discussed above such as to provide a homogeneous composition having a low viscosity. In particular, it would be especially convenient to provide a UV filter composition having well dispersed inorganic UV filter particulates while avoiding aggregates. It would be also especially convenient to provide a UV filter composition that can be easily processing to obtain different formulations, i.e. products, to fulfil the customer's requirements.

### SUMMARY OF THE INVENTION

It is an object of the present invention to obviate at least some of the disadvantages in the prior art and provide an improved UV filter composition.

Disclosed herein in a first aspect is a UV filter composition comprising:
from 35 to 80% by weight of emollient;
from 0.5 to 15% by weight of triterpenes selected from triterpene alcohols, triterpene esters, and combinations thereof; and
from 25 to 50% by weight of inorganic UV filters.

In contrary to the prior art wherein the UV filter composition comprises a very low amount of inorganic UV filters and a high quantity of emollient, it has surprisingly been found that mixing an emollient; triterpenes selected from triterpene alcohols, triterpene esters, and combinations thereof; and inorganic UV filters in the above amounts, will give a homogeneous composition having a low viscosity and a good flowability. Furthermore, since the inorganic UV filter particulates are well dispersed into the composition, the formation of aggregates is avoided. The preparation can optionally also contain a high melting wax at low concentration. This preparation is suitable for use in different beauty and personal care applications such as personal care formulation, cosmetic formulation, lip care formulation, or sun care application.

Disclosed herein in a second aspect is a formulation comprising a UV filter composition according to the present invention.

Disclosed herein in a third aspect is the use of a UV filter composition according to the present invention in a formulation.

Disclosed herein in a fourth aspect is a process of manufacturing a UV filter composition according to the present invention. Without willing to be bound by any theory, it is believed that the UV filter composition can be easily produced and processed into different formulations thanks to its low viscosity and good flowability.

### Definitions

As used herein, the term “vegetable” shall be understood as originating from a plant or a single cell organism. Thus, vegetable oil or vegetable triglycerides are still to be understood as vegetable oil or vegetable triglycerides if all the fatty acids used to obtain said triglyceride or oil is of plant or single cell organism origin.

The term “oil” as used herein refers to glyceride fats and oils containing fatty acid acyl groups and does not imply any particular melting point. The term “fat” is used synonymously with “oil” herein.

Using the nomenclature CX means that the fatty acid comprises X carbon atoms, e.g. a C14-fatty acid has 14 carbon atoms while a C16-fatty acid has 16 carbon atoms.

Using the nomenclature CX: Y means that the fatty acid comprises X carbon atoms and Y double bonds, e.g. a C14:0 fatty acid has 14 carbon atoms and 0 double bonds while a C18:1 fatty acid has 18 carbon atoms and 1 double bond.

As used herein, “%” or “percentage” relates to weight percentage i.e. wt. % or wt.-% if nothing else is indicated.

As used herein, the term “about” includes an interval around of 10% a value.

As used herein, the term “fatty acid” encompasses free fatty acids and fatty acid residues in triglycerides.

As used herein, the term “oils derived therefrom” encompasses any processed oils, i.e. oils that have undergone one or more process(es). For example, this term includes any fraction of oils, i.e. oils that have undergone fractionation. In another example, this term includes any distilled oils, i.e. oils that have undergone distillation. In another example, this term includes any oils that have undergone an alcoholysis.

Oxidation resistance as used throughout this specification and the claims is the ability to withstand oxidation and is measured as the oxidative stability index at 110° C. according to AOCS Cd 12b-92(m).

Saponification value as used throughout this specification and the claims is measured according to IUPAC 2.202.

Unsaponifiable matter value as used throughout this specification and the claims is measured according to AOCS Ca 6a-40.

Triterpene esters and/or triterpene alcohols content as used throughout this specification and the claims measured based GC-MS, using cholesterol-palmitate as standard.

Viscosity is measured using a viscometer called “Brookfield DV-E” with a type of spindle 7 at a rotation speed of 3 rotations per minute. Typically, the sample is stirred. Then, the viscosity value is determined after few seconds of stirring.

Flowability means the degree of movement of a fluid or a colloidal suspension when stirred, agitated, or otherwise disturbed from its resting state. Typically, the flowability is visually determined by pouring the sample and by evaluating the degree of movement of said sample.

Average particle size is measured using laser diffraction or dynamic light scattering.

Room temperature as used throughout the description and the claims denotes a temperature of about 20-25° C.

### DETAILED DESCRIPTION OF THE INVENTION

When describing the below embodiments, the present invention envisages all possible combinations and permutations of the below described embodiments with the above disclosed aspects.

The invention relates to a UV filter composition comprising:
from 35 to 80% by weight of emollient;
from 0.5 to 15% by weight of triterpenes selected from triterpene alcohols, triterpene esters, and combinations thereof; and
from 25 to 50% by weight of inorganic UV filters.

The present invention is based upon the surprising finding that the above UV filter composition solves or alleviates many of the problems discussed above associated as to provide homogenous and well dispersed formulation having an important content of inorganic UV filters. It has been found that the UV filter composition according to the present invention has a lower viscosity and a higher flowability when compared to an analogous UV filter composition, such as disclosed in WO9963031, that comprises less than 5% by weight of inorganic UV filters and about 1% by weight of triterpenes selected from triterpene alcohols, triterpenes esters, and combinations thereof. It is believed that the amount of the triterpenes and the amount of the inorganic UV filters play an important role. Indeed, without willing to be bound by any theory, the inventors have found that when the amount of inorganic UV filters is below less than 25%, preferably less than 15% and more preferably less than 5%, inorganic UV filters only collaborate with triterpenes to provide a UV-protecting effect. Nevertheless, such compositions have a high viscosity and a low flowability leading to a poor dispersion. Surprisingly, it has been also found that when the amount of the inorganic UV filters and the amount of the triterpenes is in the above ranges, the triterpenes provided in their purest form collaborate mainly with the UV inorganic filters to improve the dispersion of inorganic UV filters, such as titanium dioxide and/or zinc oxide, in the composition. The triterpenes alcohol and/or esters provided in a liquid form, when compared to conventional fats such as shea butter, allow an optimized mixing of all the compounds. The decrease of the viscosity in addition to the increase of the flowability have thus been observed leading to homogenous compositions.

In one or more embodiments, the triterpene alcohols are selected from Lupeol, a-amyrin, B-amyrin, Parkeol, Butyrospermol, Taraxasterol, and any combination thereof. In preferable embodiments, the triterpenes esters are selected from Lupeol cinnamate, α-amyrin cinnamate, β-amyrin cinnamate, Parkeol cinnamate, Butyrospermol cinnamate, Taraxasterol cinnamate, Lupeol acetate, a-amyrin acetate, B-amyrin acetate, Parkeol acetate, Butyrospermol acetate, Taraxasterol acetate, Lupeol oleate, α-amyrin oleate, β-amyrin oleate, Parkeol oleate, Butyrospermol oleate, Taraxasterol oleate, Lupeol stearate, α-amyrin stearate, β-amyrin stearate, Parkeol stearate, Butyrospermol stearate, Taraxasterol stearate, and any combination thereof.

In preferable embodiments, the triterpenes alcohols and/or esters are originated from shea butter unsaponifiables. Preferably, the triterpenes alcohols and/or esters are originated from shea olein unsaponifiables. The unsaponifiable part of an oil, by definition the material from a lipid sample which can be extracted by petroleum ether or diethyl ether after alkaline hydrolysis, is typically said to comprise said nonpolar to semi-polar lipids, hydrocarbons and waxes. Shea olein, has a higher content of unsaponifiable matter when compared to shea butter, for instance, up to 13%, karite hydrocarbons inclusive. The unsaponifiable lipids of shea butter are characterized by a high content of UV-absorbing triterpene esters and triterpenes alcohols having an anti-inflammatory action on damaged skin. Without willing to be bound by any theory, it is believed that the triterpenes originated from shea butter unsaponifiables, preferably form shea olein unsaponifiables, further increase the viscosity and the flowability performance of the UV filter composition. Furthermore, shea butter unsaponifiables, preferably form shea olein unsaponifiables, are considered as renewable and natural products having a high value especially in the personal care and cosmetic industry over synthetic alternatives.

In one or more embodiments, the UV filter composition comprises from 0.5 to 10% by weight of triterpenes alcohols and/or esters. Preferably, the UV filter composition comprises from 0.5 to 8% by weight of triterpenes alcohols and/or esters. For example, the UV filter composition comprises from 0.5 to 4% by weight of triterpenes alcohols and/or esters or from 4 to 8% by weight of triterpenes alcohols and/or esters.

In one or more embodiments, inorganic UV filters are selected from zinc oxide, titanium oxide and a combination thereof. Preferably, the inorganic UV filters have an average particle size of from 70 to 100 nm. In this embodiment, it is believed that inorganic UV filters further decrease the viscosity and increase the flowability of the composition.

In one or more embodiments, the UV filter composition comprises from 35 to 50%, advantageously from 40 to 50% or from 40 to 46%, by weight of inorganic UV filters. Without willing to be bound by any theory, it is believed that the above amounts of inorganic UV filters further decrease the viscosity and increase the flowability of the composition.

Preferably, the inorganic UV filters are uncoated. In other words, inorganic UV filters are not surrounded by any other compounds leading to a high UV protection.

The emollient may help to maintain the soft, smooth, and pliable appearance of skin. Emollients may be able to remain on the skin surface or in the stratum corneum to act as lubricants, to reduce flaking, and improve the skin's appearance. In one or more embodiments, the emollient is selected from a vegetable oil, a wax, a mineral oil, an ester, a glyceride, and any combination thereof.

In one or more embodiments, the vegetable oil is selected from: shea olein, cocoa butter, mango butter, illipe butter, murumuru butter, cupuacu butter, Shorea robusta seed butter, coconut oil, rapeseed oil, high oleic rapeseed oil, high erucic acid rapeseed oil, soybean oil, sunflower oil, high oleic sunflower oil, linseed oil, olive oil, corn oil, cottonseed oil, carinata oil, groundnut oil, safflower oil, high oleic safflower oil, peanut oil, avocado oil, rice oil, camelina oil, palm oil, coconut oil, oils derived therefrom and any combination thereof.

Preferably, the emollient comprises at least an oil derived from shea olein. The inventors have found that oil(s) derived from shea olein comprises a high content of triterpenes alcohols and/or esters so as to provide them in their purest liquid form. It is believed that shea olein further improves the dispersion of triterpenes alcohols and/or esters within the UV filter composition since the fluidity of said derived shea olein improves the contact surface of triterpenes with the other components of the UV filter composition. Derived shea olein may navigate easily through the whole composition entraining triterpenes so that triterpenes are dispersed homogenously all over the composition. Thus, only a low content of triterpenes alcohols and/or esters is needed to provide an improved homogeneous composition compared to conventional fats such as shea butter.

In another preferred embodiment, the emollient is free of shea butter; preferably the UV filter composition is free of shea butter. The inventors have found that using shea butter may increase the viscosity and decrease the flowability of the UV filter composition. Indeed, it is believed that shea butter being semi-solid at room temperature and comprising a low content of triterpenes alcohols and/or esters may considerably decrease the contact surface between all the components of the UV filter composition since the UV filter composition is difficult to mix.

Typically, the wax comprises a mixture of esters, hydrocarbons, fatty alcohols, fatty acids, and any combination thereof. Preferably, the wax comprises esters formed from fatty alcohols and fatty acids already present in the waxes.

Typically, the mineral oil is a complex mixture mainly consisting of saturated hydrocarbons obtained from petroleum. Mineral oil saturated hydrocarbons consist of linear and branched alkanes, and alkyl-substituted cyclo-alkanes. Preferably, the mineral oil comprises chain hydrocarbons containing from 12 to 40 carbon atoms in total.

Esters are compounds formed between acids and alcohols; glycerol being excluded. In another words, esters are non-glyceride esters. In one or more embodiments, the esters have a total carbon chain length of from 14 to 44. Advantageously, the esters are branched and/or saturated. Preferably, the esters are selected from alkyl benzoate, alkyl oleate, alkyl salicylate, alkyl palmitate, alkyl myristate, alkyl stearate, coco-caprylate/caprate, and any combination thereof. Preferably, the alkyl benzoate is C12-C15 alkyl benzoate. In another preferred embodiment, the alkyl salicylate is butyloctyl salicylate.

In one or more embodiments, the glycerides are selected from monoglycerides, diglycerides, triglycerides, and any combination thereof.

In one or more embodiments, the monoglycerides comprise of from 15 to 23 carbon atoms in total.

In one or more embodiments, the diglycerides comprise of from 23 to 41 carbon atoms in total.

In one or more embodiments, the triglycerides comprise of from 24 to 60 carbon atoms in total. Advantageously, the triglycerides are medium chain triglycerides (MCT). For example, the medium chain triglycerides are originated from palm oil or coconut oil. For example, the medium chain triglycerides comprise of from 24 to 36 carbon atoms in total.

Preferably, the UV filter composition comprises:
from 35 to 80% by weight of emollient selected from C12-C15 alkyl benzoate, butyloctyl salicylate, medium chain triglycerides, and any combination thereof;
from 0.5 to 10%, preferably from 0.5 to 8%, by weight of triterpenes selected from triterpene alcohols, triterpene esters, and combinations thereof; said triterpenes esters and/or alcohols being originated from shea butter, and preferably shea olein and
from 40 to 50%, preferably from 40 to 46 wt. %, by weight of uncoated inorganic UV filters.

The inventors have found that the above UV filter composition provides the optimal viscosity and flowability leading to a highly dispersed UV filter composition. In this embodiment, the UV filter composition has a viscosity of less than 18000 mPas, preferably less than 12000 mPa·s, more preferably of less than 5000 mPas and advantageously of less than 1000 mPa·s.

Preferably, the UV filter composition comprises:
from 35 to 80% by weight of emollient comprising at least an oil derived from shea olein;
from 0.5 to 10%, preferably from 0.5 to 8%, by weight of triterpenes selected from triterpene alcohols, triterpene esters, and combinations thereof; said triterpenes esters and/or alcohols being originated from shea butter, and preferably shea olein and
from 40 to 50%, preferably from 40 to 46 wt. %, by weight of uncoated inorganic UV filters.

In this embodiment, the UV filter composition has a viscosity of less than 17000 mPas.

In one or more embodiments, the UV filter composition has an oxidative stability index at 110° C. determined according to AOCS Cd 12b-92(m) exceeds 40 hours. It is an advantage of the invention that the composition is highly oxidation resistant. As oxidative stability is an important factor when using an emollient, the choice of ingredients is important. Using an emollient in combination with triterpenes esters and/or alcohols increases the oxidative stability to above 40 hours. In addition, antioxidants can be added to the product to increase oxidative stability. In one or more embodiments, the UV filter composition further comprises at least one antioxidant.

In one or more embodiments, the UV filter composition has a viscosity of less than 20000 mPas. Without willing to be bound by any theory, such UV filter composition provides a well dispersed and homogenous composition.

The invention also relates to a formulation comprising a UV filter composition according to present invention.

In one or more embodiments, the formulation is a personal care formulation, cosmetic formulation, lip care formulation, or a sun care application.

In one or more embodiments, the formulation is in a form of an emulsion, anhydrous formulation, cream, capsule or stick.

In one or more embodiments, the emulsion or the anhydrous formulation is sprayable.

The invention further relates to the use of a UV filter composition according to the present invention in a formulation.

Preferably, the use comprises applying the UV filter composition every 40 hours. Without willing to be bound by any theory, the UV filter composition has an oxidative stability to above 40 hours. Thus, it is possible to apply such UV filter composition every 40 hours to obtain the optimal UV protection.

In one or more embodiments, the use comprises using the UV filter composition having an improved flowability when compared to an analogous UV filter composition that comprises less than 25%, preferably less than 15% and more preferably less than 5%, by weight of inorganic UV filters and about 1% by weight of triterpenes selected from triterpene alcohols, triterpene esters, and combinations thereof.

In one or more embodiments, the use comprises using the UV filter composition to improve the viscosity when compared to an analogous UV filter composition that comprises less than 25%, preferably less than 15% and more preferably less than 5%, by weight of inorganic UV filters and about 1% by weight of triterpenes selected from triterpene alcohols, triterpenes esters, and combinations thereof.

The inventors have found that significantly increasing the amount of inorganic UV filters according to the present invention actually results in an improvement in the overall viscosity and flowability of the UV filter composition which is counterintuitive in view of the prior art that usually teaches to reduce the amounts of the UV filters to obtain a homogeneous composition.

The invention further relates to a process of manufacturing a UV filter composition according to the present invention, or a formulation according to the present invention, wherein the process comprises:
A). providing from 35 to 80% by weight of emollient; from 0.5 to 15% by weight of triterpenes selected from triterpene alcohols, triterpenes esters, or any combination thereof; and from 10 to 50% by weight of UV filters, and optionally additives; and
B). mixing the components of step A).

In one or more embodiments, in step B), the components of step A) are heated during the mixing. Preferably, the components are heated to a temperature between 3° and 100° C., such as 30, 40, 50, 60, 70, 80, 90 95, 97 or even 99° C.

Preferably, in step B), the components of step A) are not heated during the mixing. Indeed, it is believed that all the components of the UV filter compositions according to the invention collaborate to form a homogenous and well dispersed composition without the need of heating the components.

### DETAILED DESCRIPTION OF THE INVENTION

The following examples are for illustrative purposes only and are not intended to limit the scope of the invention in any way.

### EXAMPLES

### Raw Materials

Zano® 20 is an inorganic UV filter comprising particles of zinc oxide (ZnO). Lipex SheaSolve™ (Lipex SheaLight™) is a liquid triterpenes esters blend derived from shea butter, especially from shea olein, comprising between 45 and 65 wt. % of ethyl-oleate, between 5 and 10 wt. % of ethyl-linolate, between 20 and 30 wt. % of ethyl stearate, between 3 and 7 wt. % of ethyl palmitate and between 5 and 10 wt. % of shea butter unsaponifiables, which corresponds to a triterpene alcohols and triterpene esters content of 6-12 wt. %.

Lipex SheaTris™ is a liquid triterpenes esters blend derived from shea butter, especially from shea olein, comprising 57 wt. % of triterpene esters and triterpenes alcohols and 43 wt. % of glycerides. The glycerides include diglycerides and triglycerides originating from shea butter. The 57 wt. % of triterpene esters and triterpenes alcohols content corresponds to shea butter unsaponifiables of 48 wt. %.

Lipex® BC™ is a vegetable blend including. 53 wt. % of hydrogenated soy bean and 47 wt. % of hydrogenated palm oil.

Lipex® PreAct™ is hydrogenated canola oil

Lipex® SMP™ is hydrogenated palm kernel oil.

Candelilla wax is a wax derived from the leaves of the small Candelilla shrub native to northern Mexico and the southwestern United States, Euphorbia cerifera and Euphorbia antisyphilitica, from the family Euphorbiaceae.

Beeswax white is a natural wax produced by honeybees of the genus Apis.

Lipex® Shea-U™ is a liquid triterpenes esters blend derived from shea butter, especially from shea olein, comprising cinnamate esters, phytosterols, tocopherols and 20 wt. % of triterpene esters and triterpenes alcohols.

Tioveil™ OP comprises Titanium Dioxide, Cyclomethicone, C12-15 Alkyl Benzoate, Polyhydroxystearic Acid, Aluminium Stearate and Alumina.

C12-C15 Alkyl benzoate, CAS 68411-27-8, is an ester formed from benzoic acid and C12-C15 alcohols. It is a colorless oil defined by a molecular weight between 278-321 g/mole, a saponification value between 168-183 and a density of around 910-940 gram/liter at 20° C.

Ethyl oleate, CAS 111-62-6, is a fatty acid ester formed by the condensation of oleic acid and ethanol. It is a colorless oil defined by a molecular weight of 310 g/mole and a density of around 850 gram/liter at 25° C. The purity of the ethyl oleate used was 98%.

Akomed® comprises at least 99% of triglycerides formed glycerol and fatty acids including at least 95% of capric or caprylic acids.

Butyloctyl Salicylate, CAS 190085-41-7, is a branched chain ester formed from 2-butyl-1-octanol and salicylic acid. The physical state at room temperature is liquid, the saponification value of about 181, the specific gravity of 0.974 and a refractive index of around 1.492.

### Experimental Procedure

The following procedure was used for the preparation of UV filter compositions of the following examples 1 to 16:
A). The emollient; the triterpenes selected from triterpene alcohols, triterpenes esters, and any combination thereof; and the inorganic UV filters, here zinc oxide (ZnO) were provided;
B). the components of step A) were mixed using a dispersion tool called “TM50 Three Roll Mill”. First rollers were rotating at a speed of 60 rpm and second rollers were rotating at a speed of 80 rpm. The UV filter compositions were passed three times in the dispersion tool.

The procedure according to WO9963031 was used for the preparation of UV filter compositions of the following examples 17 and 18:
1) Three phases were produced, phases A, B and C.

### Phase A

Table 1 shows the ingredients of phase A.

TABLE 1

| Ingredients | INCI name | Grams |
|---|---|---|
| Lipex ® BC ™ | Hydrogenated vegetable oil | 70 |
| Lipex ® SMP ™ | Hydrogenated palm kernel oil | 30 |
| Candelilla wax | Candelilla wax | 12 |
| Beeswax white | Beeswax | 8 |
| Akogel ™ | Hydrogenated vegetable oil | 14 |

All ingredients of phase A were completely melted at 85° C. and blended in a 500 ml beaker. After blending, phase A was divided and transferred into two new beakers, containing each 52 grams of phase A.

The two beakers were kept at 85° C.

### Phase B

Table 2 shows the ingredients of phase B.

TABLE 2

| Ingredients | INCI name | Grams |
|---|---|---|
| Akomed ® | Caprylic/capric triglycerides | 23 |
| Lipex ® PreAct ™ | Hydrogenated Canola Oil | 4 |
| Lipex ® Shea-U ™ | Butyrospermum Parkii Extract | 4 |
| Tioveil ™ OP | Ethylhexyl Palmitate (and) | 2 |
| | Titanium Dioxide (and) | |
| | Alumina (and) | |
| | Polyhydroxystearic Acid | |
| | (and) Silica | |

All ingredients of Phase B were melted and blended at 40° C. and kept at 40° C.

### Phase C

Table 3 shows the ingredients of phase C.

TABLE 3

| Ingredients | INCI name | Grams |
|---|---|---|
| Akomed ® | Caprylic/capric triglycerides | 27 |
| Lipex ® PreAct ™ | Hydrogenated Canola Oil | 4 |
| Lipex ® Shea-U ™ | Shea Butter | 0 |
| Tioveil ™ OP | Ethylhexyl Palmitate (and) | 2 |
| | Titanium Dioxide (and) | |
| | Alumina (and) | |
| | Polyhydroxystearic Acid | |
| | (and) Silica | |

All ingredients of Phase C were melted and blended at 40° C. and kept at 40° C.
2) Mixing of phases A and B (Example 17), and A and C (Example 18).

52 grams of phase A (temperature 85° C.) was poured into Phase B beaker (40° C.). 52 grams of phase A (temperature 85° C.) was poured into Phase C beaker (40° C.). Both blends were mixed thoroughly. After mixing, the blends were transferred to a mould and cooled at room temperature for two hours.

Then, all the UV filter compositions (Examples 1 to 18) were rested for at least 48 hours at 20° C. The UV filter compositions were stirred for 10-20 seconds. Then, the viscosity and the flowability were measured according to the herein described methods. The flowability has been rated according to the following rating scale: 0 means that no flow is present, i.e. there was no movement of the composition when pouring it, 1 means that the flowability is very poor, 2 means that the flowability is poor, 3 means that the flowability is acceptable, 4 means that the flowability good and 5 means that the flowability is very good.

TABLE 1

| | Components | | | | | |
|---|---|---|---|---|---|---|
| | | | triterpene | Uncoated | | |
| | | | alcohols | inorganic | | |
| | | | and | UV filters - | Results | |
| | | | triterpene | ZnO | Viscosity | |
| Examples | Emollients (wt. %) | | esters | (Zano ® 20) | (mPas) | Flowability |
| 1 | 60% | — | — | 40% | 24000 | 2 |
| | Butyloctyl | | | | | |
| | Salicylate | | | | | |
| 2* | 58% | 2% Lipex SheaTris ™ | | 40% | 500 | 5 |
| | Butyloctyl | 0.8% | 1.2% | | | |
| | Salicylate | glycerides | | | | |
| 3* | 55% | 5% Lipex SheaTris ™ | | 40% | 12000 | 4 |
| | Butyloctyl | 2.2% | 2.8% | | | |
| | Salicylate | glycerides | | | | |
| 4 | 50% | — | — | 50% | Impossible | 0 |
| | Butyloctyl | | | | to measure | |
| | Salicylate | | | | | |
| 5 | 60% | — | — | 40% | 33,000 | 2 |
| | C12-15 Alkyl | | | | | |
| | Benzoate | | | | | |
| 6* | 59% | 1% Lipex SheaTris ™ | | 40% | 4000 | 4 |
| | C12-15 Alkyl | 0.4% | 0.6% | | | |
| | Benzoate | glycerides | | | | |
| 7* | 58% | 2% Lipex SheaTris ™ | | 40% | 500 | 5 |
| | C12-15 Alkyl | 0.8% | 1.2% | | | |
| | Benzoate | glycerides | | | | |
| 8* | 55% | 5% Lipex SheaTris ™ | | 40% | 11000 | 4 |
| | C12-15 Alkyl | 2.2% | 2.8% | | | |
| | Benzoate | glycerides | | | | |
| 9 | 50% | — | — | 50% | Impossible | 0 |
| | C12-15 Alkyl | | | | to measure | |
| | Benzoate | | | | | |
| 10* | 53% | 2% Lipex SheaTris ™ | | 40% | 500 | 5 |
| | C12-15 Alkyl | 0.8% | 1.2% | | | |
| | Benzoate + | glycerides | | | | |
| | 5% mineral | | | | | |
| | oil | | | | | |
| 11* | 60% Lipex SheaLight ™ | | | 40% | 6000 | 4 |
| | 52.6% | 0.2% | 7.2% | | | |
| | ethyl-oleate, | glycerides | | | | |
| | ethyl-linolate, | | | | | |
| | ethyl stearate, | | | | | |
| | ethyl palmitate | | | | | |
| 12* | 52% Lipex SheaLight ™ + | | | 46% | 15000 | 4 |
| | 2% Lipex SheaTris ™ | | | | | |
| | 44.8% | 1% | 8.2% | | | |
| | ethyl-oleate, | glycerides | | | | |
| | ethyl-linolate, | | | | | |
| | ethyl stearate, | | | | | |
| | ethyl palmitate | | | | | |
| 13 | 54% | — | — | 46% | 35000 | 2 |
| | Ethyl oleate | | | | | |
| 14* | 52% | 2% Lipex SheaTris ™ | | 46% | 17000 | 3 |
| | Ethyl oleate | 0.8% | 1.2% | | | |
| | | glycerides | | | | |
| 15 | 50% | — | — | 50% | 51000 | 1 |
| | Ethyl oleate | | | | | |
| 16 | 60% | — | — | 40% | 41000 | 1 |
| | Akomed R ™M | | | | | |
| 17 | 94% | 4% Lipex ® Shea-U ™ | | <2% TiO₂ | Impossible | 0 |
| | See Tables 1 | 1.1 wt. % | 2.9 | | to measure | |
| | and 2 | | | | | |
| 18 | 94% | — | — | <2% TiO₂ | Impossible | 0 |
| | See Tables 1 | | | | to measure | |
| | and 3 | | | | | |

*according to the present invention

Comparative Examples 1, 5, 13, 15, 16 were very thick. Furthermore, comparative Examples 1, 5, 13, 15, 16 had a high viscosity and a low flowability. Examples 2, 3, 6, 7, 8, 10, 11, 12 and 14 had a lower viscosity and a higher flowability when compared to the Comparative Examples. Examples 2, 3, 6, 7, 8, 10, 11, 12 and 14 were homogenous and well dispersed formulation even with an important content of inorganic UV filters. It is believed that when the amount of ZnO and the amount of the triterpenes is in the ranges of Examples 2, 3, 6, 7, 8, 10, 11, 12 and 14, the triterpenes collaborate mainly with the UV inorganic filters to improve the dispersion of said ZnO. Comparatives Examples 4, 9, 17 and 18 were too hard to measure the viscosity, hence it was not possible to achieve a value. Furthermore, it was impossible to pour Examples 4, 9, 17 and 18, hence the flowability was scored non-flowable. Examples 17 and 18 clearly show that when the content of the inorganic UV filters is very low, the presence of triterpenes esters and/or alcohols does not have any impact on the viscosity and the flowability of the UV filter composition.

1. A UV filter composition comprising:
from 35 to 80% by weight of emollient;
from 0.5 to 15% by weight of triterpenes selected from triterpene alcohols, triterpene esters, and combinations thereof; and
from 25 to 50% by weight of inorganic UV filters.

2. The UV filter composition according to claim 1, wherein the triterpene alcohols are selected from Lupeol, α-amyrin, β-amyrin, Parkeol, Butyrospermol, Taraxasterol, and any combination thereof.

3. The UV filter composition according to claim 1, wherein the triterpenes esters are selected from Lupeol cinnamate, α-amyrin cinnamate, β-amyrin cinnamate, Parkeol cinnamate, Butyrospermol cinnamate, Taraxasterol cinnamate, Lupeol acetate, α-amyrin acetate, β-amyrin acetate, Parkeol acetate, Butyrospermol acetate, Taraxasterol acetate, Lupeol oleate, α-amyrin oleate, β-amyrin oleate, Parkeol oleate, Butyrospermol oleate, Taraxasterol oleate, Lupeol stearate, α-amyrin stearate, β-amyrin stearate, Parkeol stearate, Butyrospermol stearate, Taraxasterol stearate, and any combination thereof.

4. The UV filter composition according to claim 1, wherein the triterpenes alcohols and/or esters are originated from shea butter unsaponifiables.

5. The UV filter composition according to claim 1, wherein the UV filter composition comprises from 0.5 to 10% by weight of triterpenes alcohols and/or esters.

6. The UV filter composition according to claim 1, wherein the UV filter composition comprises from 0.5 to 8% by weight of triterpenes alcohols and/or esters.

7. The UV filter composition according to claim 1, wherein inorganic UV filters are selected from zinc oxide, titanium oxide and a combination thereof.

8. The UV filter composition according to claim 1, wherein the UV filter composition comprises from 35 to 50% by weight of inorganic UV filters.

9. The UV filter composition according to claim 1, wherein the inorganic UV filters are uncoated.

10. The UV filter composition according to claim 1, wherein the emollient is selected from a vegetable oil, a wax, a mineral oil, an ester, a glyceride, and any combination thereof.

11. The UV filter composition according to claim 10, wherein the vegetable oil is selected from shea olein, cocoa butter, mango butter, illipe butter, murumuru butter, cupuacu butter, and Shorea robusta seed butter, coconut oil, rapeseed oil, high oleic rapeseed oil, high erucic acid rapeseed oil, soybean oil, sunflower oil, high oleic sunflower oil, linseed oil, olive oil, corn oil, cottonseed oil, carinata oil, groundnut oil, safflower oil, high oleic safflower oil, peanut oil, avocado oil, rice oil, camelina oil, palm oil, coconut oil, oils derived therefrom and any combination thereof.

12. The UV filter composition according to claim 1, wherein the emollient comprises at least an oil derived from shea olein.

13. The UV filter composition according to claim 1, wherein the emollient is free of shea butter.

14. The UV filter composition according to claim 10, wherein the wax comprises a mixture of esters, hydrocarbons, fatty alcohols, fatty acids, or any combination thereof.

15. The UV filter composition according to claim 10, wherein the mineral oil comprises chain hydrocarbons containing from 12 to 40 carbon atoms in total.

16. The UV filter composition according to claim 10, wherein the ester has esters have a total carbon chain length of from 14 to 44.

17. The UV filter composition according to claim 10, wherein the ester is selected from alkyl benzoate, alkyl oleate, alkyl salicylate, alkyl palmitate, alkyl myristate, alkyl stearate, coco-caprylate/caprate, and any combination thereof.

18. The UV filter composition according to claim 17, wherein the alkyl benzoate is C12-C15 alkyl benzoate.

19. The UV filter composition according to claim 17, wherein the alkyl salicylate is butyloctyl salicylate.

20. The UV filter composition according to claim 10, wherein the glyceride is selected from monoglycerides, diglycerides, triglycerides, and any combination thereof.

21. The UV filter composition according to claim 20, wherein the monoglycerides comprise of from 15 to 23 carbon atoms in total.

22. The UV filter composition according to claim 20, wherein the diglycerides comprise of from 23 to 41 carbon atoms in total.

23. The UV filter composition according to claim 20, wherein the triglycerides comprise of from 24 to 60 carbon atoms in total.

24. The UV filter composition according to claim 20, wherein triglycerides are medium chain triglycerides.

25. The UV filter composition according to claim 1, wherein UV filter composition comprises:
from 35 to 80% by weight of emollient selected from C12-C15 alkyl benzoate, butyloctyl salicylate, medium chain triglycerides, and any combination thereof;
from 0.5 to 10%, and
from 40 to 50%, of uncoated inorganic UV filters.

26. The UV filter composition according to claim 1, wherein UV filter composition comprises:
from 35 to 80% by weight of emollient comprising at least an oil derived from shea olein;
from 0.5 to 10% of triterpenes selected from triterpene alcohols, triterpene esters, and combinations thereof; said triterpenes esters and/or alcohols being originated from shea butter, and
from 40 to 50% by weight of uncoated inorganic UV filters.

27. The UV filter composition according to claim 1, wherein the UV filter composition has an oxidative stability index at 110° C. determined according to AOCS Cd 12b-92(m) exceeds 40 hours.

28. The UV filter composition according to claim 1, wherein the UV filter composition has a viscosity of less than 20000 mPas.

29. A formulation comprising a UV filter composition according to claim 1.

30. The formulation according to claim 29, wherein the formulation is a personal care formulation, cosmetic formulation, lip care formulation, or a sun care application.

31. The formulation according to claim 29, wherein the formulation is in a form of an emulsion, anhydrous-formulation, cream, capsule or stick.

32. The formulation according to claim 31, wherein the emulsion or the anhydrous formulation is sprayable.

33. (canceled)

34. (canceled)

35. (canceled)

36. (canceled)

37. A process of manufacturing a UV filter composition according to any one of claim 1, wherein the process comprises:
A). providing from 35 to 80% by weight of emollient; from 0.5 to 15% by weight of triterpenes selected from triterpene alcohols, triterpenes esters, or any combination thereof; and from 25 to 50% by weight of UV filters; and
B). mixing the components of step A).
